(19) United States
(12) Patent Application Publication
(10) Pub. No.: US 2024/0316009 A1
(43) Pub. Date: Sep. 26, 2024

(54) TINOSTAMUSTINE FOR USE IN THE TREATMENT OF T-CELL PROLYMPHOCYTIC LEUKAEMIA

(71) Applicant: Purdue Pharma L.P., Stamford, CT (US)

(72) Inventors: Thomas Jorg Mehrling, Riehen (CH); Marco Herling, Cologne (DE)

(73) Assignee: Purdue Pharma L.P., Stamford, CT (US)

(21) Appl. No.: 18/422,118

(22) Filed: Jan. 25, 2024

Related U.S. Application Data
(63) Continuation of application No. 17/730,276, filed on Apr. 27, 2022, now Pat. No. 11,918,558.
Application No. 17/730,276 is a continuation of application No. 16/621,898, filed on Dec. 12, 2019, now Pat. No. 11,318,117.

(30) Foreign Application Priority Data
Jun. 13, 2017 (GB) 1709402.0

(51) Int. Cl. A61K 31/4184; A61K 9/00; A61P 35/02

(52) U.S. Cl. CPC A61K 31/4184; A61K 9/0019; A61P 35/02

(57) ABSTRACT

There is provided tinostamustine or a pharmaceutically acceptable salt thereof for use in the treatment of T-cell prolymphocytic leukemia (T-PLL) in a patient in need thereof.

### CROSS-REFERENCE TO RELATED APPLICATIONS

This application is a continuation application of U.S. patent application Ser. No. 17/730,276, filed on Apr. 27, 2022, which is a continuation application of U.S. patent application Ser. No. 16/621,898, filed on Dec. 12, 2019, now U.S. Pat. No. 11,318,117, issued on May 3, 2022, which is a U.S. national stage application filed under 35 U.S.C. 371 based on International Patent Application No. PCT/EP2018/065664, filed on Jun. 13, 2018, which claims priority to Great Britain Application No. 1709402.0, filed on Jun. 13, 2017. The contents of each of the aforementioned applications are herein incorporated by reference in their entities.

### TECHNICAL FIELD

The present invention relates to a method of treating T-cell prolymphocytic leukemia (T-PLL).

### BACKGROUND TO THE INVENTION

Cancer is one of the most life threatening diseases. Cancer is a condition in which cells in a part of the body experience out-of-control growth. According to latest data from American Cancer Society, it is estimated there will be 1.69 million new cases of cancer in USA in 2017. Cancer is the second leading cause of death in the United States (second only to heart disease) and will claim more than 601,000 lives in 2017. In fact, it is estimated the average lifetime risk of developing cancer is 40.8% for American males and 37.5% for American women. Therefore cancer constitutes a major public health burden and represents a significant cost in the United States. These figures are reflected elsewhere across most countries globally, although the types of cancer and relative proportions of the population developing the cancers vary depending upon many different factors such including genetics and diet.

For decades surgery, chemotherapy, and radiation were the established treatments for various cancers. Patients usually receive a combination of these treatments depending upon the type and extent of their disease. But chemotherapy is the most important option for cancer patients when surgical treatment (i.e. the removal of diseased tissue) is impossible. While surgery is sometimes effective in removing tumours located at certain sites, for example, in the breast, colon, and skin, it cannot be used in the treatment of tumours located in other areas, such as the backbone, nor in the treatment of disseminated hematologic cancers include cancers of the blood and blood-forming tissues (such as the bone marrow). They include multiple myeloma, lymphoma and leukemia. Radiation therapy involves the exposure of living tissue to ionizing radiation causing death or damage to the exposed cells. Side effects from radiation therapy may be acute and temporary, while others may be irreversible. Chemotherapy involves the disruption of cell replication or cell metabolism. It is used most often in the treatment of breast, lung, and testicular cancer. One of the main causes of failure in this treatment of cancer is the development of drug resistance by the cancer cells, a serious problem that may lead to recurrence of disease or even death. Thus, more effective cancer treatments are needed.

Leukemia is a cancer of the blood cells. Leukemias begin in the blood-forming tissue of the bone marrow. The cancers do not form solid tumours but instead large numbers of abnormal white blood cells (leukemia cells and leukemic blast cells) build up in the blood and bone marrow. There are four main types of leukemia: Acute myeloid leukemia (AML), Chronic myeloid leukemia (CML), Acute lymphocytic leukemia (ALL), and Chronic lymphocytic leukemia (CLL).

T-cell prolymphocytic leukemia (T-PLL) is recognised in the WHO classification of hematologic malignancies as a leukemic peripheral T-cell neoplasm and is of mature T-cell phenotype. Although representing the most frequent mature T-cell leukemia, T-PLL is nevertheless an extremely uncommon hematological malignancy and is rarely encountered in daily routine (with incidence of ˜0.6/million). T-PLL also has a very poor prognosis, with the median overall survival of patients with T-PLL being around 7 months with conventional chemotherapy.

Patients with T-PLL typically present with exponentially rising lymphocyte counts in peripheral blood accompanied by lymphadenopathy with hepatosplenomegaly, and bone marrow involvement.

T-PLL characteristically shows rapid progression and does not respond well to standard multi-agent chemotherapy. The monoclonal anti-CD52 antibody alemtuzumab was the only (targeted) agent that was shown to induce a high rate of remission, albeit with relapse the rule. Alemtuzumab had overall response rates ranging from 51-95% with a median survival of 15-19 months in patients achieving a complete response.

However, alemtuzumab was withdrawn from the market in 2012 and there is currently no effective first line treatment for T-PLL.

There is therefore a need for effective chemotherapeutic treatments of T-PLL.

In WO-A-2010/085377, the compound of formula I below is disclosed. It is a first-in-class dual-functional alkylating-HDACi fusion molecule which potently inhibits the HDAC pathway.

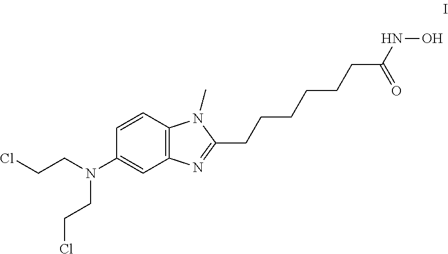 [SMILES: Cn1c(CCCCCCC(=O)NO)nc2cc(N(CCCl)CCCl)ccc21]

Biological assays showed that the compound of formula I potently inhibits HDAC enzyme (HDAC1 IC₅₀ of 9 nM). The compound of formula I has an INN of tinostamustine and is also known in the art as EDO-S101. It is an AK-DAC (a first-in-class alkylating deacetylase molecule) that, in preclinical studies, has been shown to simultaneously improve access to the DNA strands within cancer cells, break them and block damage repair.

### SUMMARY OF THE INVENTION

In a first aspect of the present invention there is provided tinostamustine or a pharmaceutically acceptable salt thereof for use in the treatment of T-cell prolymphocytic leukemia (T-PLL).

It has surprisingly been discovered that tinostamustine or a pharmaceutically acceptable salt thereof is particularly effective in the treatment of T-PLL, with activity data showing strong in vitro and in vivo sensitivity to this compound. Thus, the need for a new and effective treatment of T-PLL is met by the present invention.

In a further aspect of the present invention there is provided use of tinostamustine or a pharmaceutically acceptable salt thereof for the manufacture of a medicament for the treatment of T-PLL.

In a further aspect of the present invention there is provided a method of treating T-PLL in a patient in need thereof comprising administering to said patient an effective amount of tinostamustine or a pharmaceutically acceptable salt thereof.

In a further aspect of the present invention there is provided a kit comprising tinostamustine or a pharmaceutically acceptable salt thereof together with instructions for treating T-PLL.

The following features apply to all aspects of the invention.

### DESCRIPTION OF THE DRAWINGS

FIG. 1a shows a plot of HH cell viability relative to control after exposure to increasing concentrations of the HDAC inhibitor SAHA (vorinostat), bendamustine, SAHA+bendamustine, and EDO-S101;

FIG. 1b shows a dose response curve for the SAHA (vorinostat), bendamustine, SAHA+bendamustine, and EDO-S101, in HH cells.

FIG. 2 shows western blots analysis of patient T-PLL samples showing the effect of SAHA (vorinostat), bendamustine, SAHA+bendamustine, and EDO-S101 on various markers relevant to T-PLL.

FIG. 3a is a dose response curve showing the relative number of living T-PLL cells in suspension culture after 48 h treatment comparing SAHA (vorinostat), bendamustine, SAHA+bendamustine, and EDO-S101;

FIG. 3b shows the effect of increasing concentrations of SAHA (vorinostat), bendamustine, SAHA+bendamustine, and EDO-S101, on primary T-PLL cells with and without co-cultures of the human bone marrow stromal cell line NKtert.

FIG. 3c shows the effect of increasing concentrations of SAHA (vorinostat), bendamustine, SAHA+bendamustine, and EDO-S101, on NKtert cell viability.

FIG. 3d shows reduction in viability of NKtert cells treated with EDO-S101, vorinostat, or an equimolar combination of vorinostat and bendamustine.

FIGS. 4a 4b and 4c show the results of a transfer model for CD2-MTCP1 p13 mice investigating fludarabine, bendamustine and EDO-S101.

FIG. 5 shows the results of a transfer model for ΔJAK1 mice investigating fludarabine, bendamustine and EDO-S101.

### DETAILED DESCRIPTION OF THE INVENTION

In the present application, a number of general terms and phrases are used, which should be interpreted as follows.

The compound of formula I has an INN of tinostamustine and is also known in the art as EDO-S101. The IUPAC name is 7-(5-(bis(2-chloroethyl)amino)-1-methyl-1H-benzo[d]imidazol-2-yl)-N-hydroxyheptanamide.

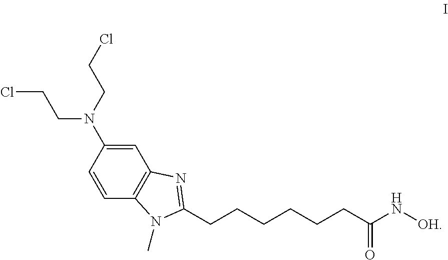 [SMILES: Cn1c(CCCCCCC(=O)NO)nc2cc(N(CCCl)CCCl)ccc21]

“Patient” includes humans, non-human mammals (e.g., dogs, cats, rabbits, cattle, horses, sheep, goats, swine, deer, and the like) and non-mammals (e.g., birds, and the like).

“Pharmaceutically acceptable salts” means salts of compounds of the present invention which are pharmaceutically acceptable, as defined above, and which possess the desired pharmacological activity. Such salts include acid addition salts formed with inorganic acids, or with organic acids. Pharmaceutically acceptable salts also include base addition salts which may be formed when acidic protons present are capable of reacting with inorganic or organic bases. Generally, such salts are, for example, prepared by reacting the free acid or base forms of these compounds with a stoichiometric amount of the appropriate base or acid in water or in an organic solvent or in a mixture of the two. Generally, non-aqueous media like ether, ethyl acetate, ethanol, isopropanol or acetonitrile are preferred. Examples of the acid addition salts include mineral acid addition salts such as, for example, hydrochloride, hydrobromide, hydroiodide, sulfate, bisulfate, sulfamate, nitrate, phosphate, and organic acid addition salts such as, for example, acetate, trifluoroacetate, maleate, fumarate, citrate, oxalate, succinate, tartrate, salicylate, tosylate, lactate, naphthalenesulphonae, malate, mandelate, methanesulfonate and p-toluenesulfonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine and basic aminoacids salts.

In the present invention, the pharmaceutically acceptable salt of tinostamustine may preferably be the hydrochloride, hydrobromide, hydroiodide, sulfate, bisulfate, sulfamate, nitrate, phosphate, citrate, methanesulfonate, trifluoroacetate, glutamate, glucuronate, glutarate, malate, maleate, oxalate, succinate, fumarate, tartrate, tosylate, mandelate, salicylate, lactate, p-toluenesulfonate, naphthalenesulfonate or acetate salt.

It has been found that tinostamustine or a pharmaceutically acceptable salt thereof shows surprising efficacy in T-PLL. In particular, it has been found that tinostamustine or a pharmaceutically acceptable salt thereof is useful in the treatment of T-PLL.

T-cell prolymphocytic leukemia or T-PLL is a leukemic peripheral T-cell neoplasm and is of mature T-cell phenotype (Campo, E et al, Blood 117, 2011 5019-32). Although representing the most frequent mature T-cell leukemia, T-PLL is nevertheless an extremely uncommon hematological malignancy and is rarely encountered in daily routine (with incidence of ˜0.6/million). T-PLL also has a very poor prognosis, with the median overall survival of patients with T-PLL being around 7 months with conventional chemotherapy.

Patients with T-PLL typically present with exponentially rising lymphocyte counts in peripheral blood accompanied by lymphadenopathy with hepatosplenomegaly, and bone marrow involvement.

The therapeutically effective amount of tinostamustine or a pharmaceutically acceptable salt administered to the patient is an amount which confers a therapeutic effect in accordance with the present invention on the treated subject, at a reasonable benefit/risk ratio applicable to any medical treatment. The therapeutic effect may be objective (i.e. measurable by some test or marker) or subjective (i.e. subject gives an indication of or feels an effect). An effective amount of tinostamustine or a pharmaceutically acceptable salt thereof according to the present invention is believed to be one wherein tinostamustine or a pharmaceutically acceptable salt thereof is included at a dosage range of from 0.3 mg/m² to 300 mg/m² body surface area of the patient or from 20 mg/m² to 150 mg/m² body surface area of the patient.

The specific therapeutically effective dose level for any particular patient will depend upon a variety of factors including the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

“Metastatic Cancer”. Cancer has the ability to spread within the body. Cancer cells can spread locally by moving into nearby normal tissue. Cancer can also spread regionally, to nearby lymph nodes, tissues, or organs. Cancer can therefore spread to distant parts of the body. When this happens, it is called metastatic cancer (also known as stage IV cancer), and the process by which cancer cells spread to other parts of the body is called metastasis. Thus, in metastasis, cancer cells break away from where they first formed (primary cancer), travel through the blood or lymph system, and form new tumours (metastatic tumours) in other parts of the body.

Metastatic cancer cells have features like that of the primary cancer and not like the cells in the place where the cancer is found. This enables doctors to tell whether a cancer is metastatic. Metastatic cancers are given the same name as the primary cancer. For example, breast cancer that has spread to the lung is called metastatic breast cancer, not lung cancer. It is treated as stage IV breast cancer, not as lung cancer.

Metastatic T-PLL refers to a T-cell prolymphocytic leukemia that has metastasised to a new location in the body. The cancer is treated as a stage IV T-PLL cancer.

“Advanced Cancer” is a cancer that is not curable but responds to treatment. Disease directed therapy is still very important because it prolongs life. For terminal cancer, therapy cannot prolong survival significantly due to the progressive nature of the disease and palliative care is the main treatment option.

Suitable examples of the administration form of tinostamustine or a pharmaceutically acceptable salt thereof include without limitation oral, topical, parenteral, sublingual, rectal, vaginal, ocular, and intranasal. Parenteral administration includes subcutaneous injections, intravenous, intramuscular, intrasternal injection or infusion techniques. Preferably, tinostamustine or a pharmaceutically acceptable salt thereof is administered parenterally, and most preferably intravenously.

Preferably, tinostamustine or a pharmaceutically acceptable salt thereof is administered intravenously to the patient in need thereof at a dosage level to the patient in need thereof of from 0.3 mg/m² to 300 mg/m² body surface area of the patient.

Preferably, tinostamustine or a pharmaceutically acceptable salt thereof is administered intravenously to the patient in need thereof at a dosage level to the patient in need thereof of from 20 mg/m² to 150 mg/m² body surface area of the patient.

It has been found that in embodiments of the present invention, tinostamustine or a pharmaceutically acceptable salt thereof or medicament comprising the same may preferably be administered to a patient in need thereof on day 1 of each treatment cycle.

Tinostamustine or a pharmaceutically acceptable salt thereof may be administered on day 1 of a 21 day treatment cycle.

In embodiments according to the present invention, tinostamustine or a pharmaceutically acceptable salt thereof or medicament comprising the same is administered to a patient in need thereof over an infusion time of 60 minutes; or an infusion time of 45 minutes; or an infusion time of 30 minutes.

In embodiments according to the present invention, tinostamustine or a pharmaceutically acceptable salt is administered to the patient in need thereof at a dosage level of from 20 mg/m² to 150 mg/m² body surface area of the patient, on day 1 of a 21 day treatment cycle, over an infusion time of 60 minutes.

In embodiments of the present invention, there is provided a kit comprising tinostamustine or a pharmaceutically acceptable salt thereof together with instructions for treating T-PLL.

The instructions may advise administering tinostamustine or a pharmaceutically acceptable salt thereof according to variables such as the state of the T-PLL being treated; the age, body weight, general health, sex and diet of the patient; the time of administration, route of administration, and rate of excretion of the specific compounds employed; the duration of the treatment; drugs used in combination or contemporaneously with the specific compounds employed; and like factors well known in the medical arts.

In a further embodiment of the present invention, the patient in need of said treatment is given radiotherapy with (including prior to, during or after) treatment of the T-PLL with tinostamustine or a pharmaceutically acceptable salt thereof. In embodiments of the present invention, the patient is treated with tinostamustine or a pharmaceutically acceptable salt thereof and radiotherapy. Preferably, the patient is given radiotherapy treatment prior to the treatment with tinostamustine or a pharmaceutically acceptable salt thereof. The radiotherapy may be given at a dose of 1 to 5 Gy over 5-10 consecutive days and preferably 2 Gy over 5-10 consecutive days.

In a further embodiment of the present invention, the patient in need of said treatment is given radiotherapy prior to or after treatment of the T-PLL with tinostamustine or a pharmaceutically acceptable salt thereof. Preferably, the patient is given radiotherapy treatment prior to the treatment with tinostamustine or a pharmaceutically acceptable salt thereof. The radiotherapy may be given at a dose of 1 to 5 Gy over 5-10 consecutive days and preferably 2 Gy over 5-10 consecutive days.

When intended for oral administration, tinostamustine or a pharmaceutically acceptable salt thereof or medicament comprising the same may be in solid or liquid form, where semi-solid, semi-liquid, suspension and gel forms are included within the forms considered herein as either solid or liquid.

Tinostamustine or a pharmaceutically acceptable salt thereof or medicament comprising the same can be prepared for administration using methodology well known in the pharmaceutical art. Examples of suitable pharmaceutical formulations and carriers are described in “Remington's Pharmaceutical Sciences” by E. W. Martin.

As a solid composition for oral administration, tinostamustine or a pharmaceutically acceptable salt thereof can be formulated into a powder, granule, compressed tablet, pill, capsule, chewing gum, wafer or the like form. Such a solid composition typically contains one or more inert diluents or carriers. Any inert excipient that is commonly used as a carrier or diluent may be used in compositions of the present invention, such as sugars, polyalcohols, soluble polymers, salts and lipids. Sugars and polyalcohols which may be employed include, without limitation, lactose, sucrose, mannitol, and sorbitol. Illustrative of the soluble polymers which may be employed are polyoxyethylene, poloxamers, polyvinylpyrrolidone, and dextran. Useful salts include, without limitation, sodium chloride, magnesium chloride, and calcium chloride. Lipids which may be employed include, without limitation, fatty acids, glycerol fatty acid esters, glycolipids, and phospholipids.

In addition, one or more of the following can be present: binders such as carboxymethylcellulose, ethyl cellulose, microcrystalline cellulose, or gelatin; excipients such as starch, lactose or dextrins, disintegrating agents such as alginic acid, sodium alginate, corn starch and the like; lubricants such as magnesium stearate; glidants such as colloidal silicon dioxide; sweetening agents such as sucrose or saccharin; a flavoring agent such as peppermint, methyl salicylate or orange flavoring; and a coloring agent.

When tinostamustine or a pharmaceutically acceptable salt thereof compositions is in the form of a capsule (e.g. a gelatin capsule), it can contain, in addition to materials of the above type, a liquid carrier such as polyethylene glycol, cyclodextrin or a fatty oil.

Tinostamustine or a pharmaceutically acceptable salt thereof compositions can be in the form of a liquid, e.g. an elixir, syrup, solution, emulsion or suspension. The liquid can be useful for oral administration or for delivery by injection. When intended for oral administration, tinostamustine or a pharmaceutically acceptable salt thereof compositions can comprise one or more of a sweetening agent, preservatives, dye/colorant and flavor enhancer. In tinostamustine or a pharmaceutically acceptable salt thereof compositions for administration by injection, one or more of a surfactant, preservative, wetting agent, dispersing agent, suspending agent, buffer, stabilizer and isotonic agent can also be included.

The preferred route of administration is parenteral administration including, but not limited to, intradermal, intramuscular, intraperitoneal, intravenous, subcutaneous, intranasal, epidural, intranasal, intracerebral, intraventricular, intrathecal, intravaginal or transdermal. The preferred mode of administration is left to the discretion of the practitioner, and will depend in part upon the site of the medical condition (such as the site of cancer). In a more preferred embodiment, tinostamustine or a pharmaceutically acceptable salt thereof or medicament comprising the same is administered intravenously.

Liquid forms of tinostamustine or a pharmaceutically acceptable salt thereof or medicament comprising the same, may be solutions, suspensions or other like form, and can also include one or more of the following: sterile diluents such as water for injection, saline solution, preferably physiological saline, Ringer's solution, isotonic sodium chloride, fixed oils such as synthetic mono or digylcerides, polyethylene glycols, glycerin, or other solvents; antibacterial agents such as benzyl alcohol or methyl paraben; and agents for the adjustment of tonicity such as sodium chloride or dextrose. A parenteral combination or composition can be enclosed in an ampoule, a disposable syringe or a multiple-dose vial made of glass, plastic or other material. Physiological saline is a preferred adjuvant.

Tinostamustine or a pharmaceutically acceptable salt thereof or medicament comprising the same can be administered by any convenient route, for example by infusion or bolus injection, by absorption through epithelial or mucocutaneous linings, and preferably by bolus.

Examples of compositions comprising tinostamustine or a pharmaceutically acceptable salt thereof are disclosed in WO2013/040286.

The present invention may be further understood by consideration of the following non-limiting examples.

### EXAMPLES

In the following examples, the compound having the following formula I is referred to as EDO-S101.

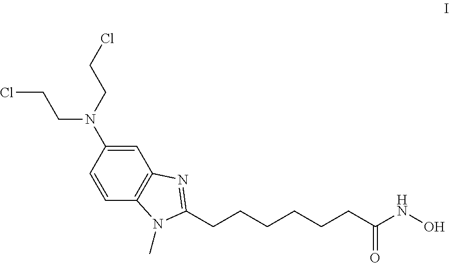 [SMILES: Cn1c(CCCCCCC(=O)NO)nc2cc(N(CCCl)CCCl)ccc21]

EDO-S101 may be prepared as described in Example 6 of WO-A-2010/085377.

### Materials and Methods

### EDO-S101 and Control Compounds

EDO-S101 was provided by EDO MundiPharma, and synthesised as described in Example 6 of WO-A-2010/085377.

Bendamustine was provided by EDO MundiPharma.

Vorinostat (SAHA) (catalogue reference number SML0061-5 mg) and Fludarabine were purchased from Sigma-Aldrich.

### Cell Culture

RPMI-1640 medium (Sigma-Aldrich) supplemented with 1% L-Glutamine (200 mM; Sigma-Aldrich), 10% fetal bovine serum (FBS) (Sigma-Aldrich) and Penicillin/Streptomycin (100 U/0.1M; PAA) was used for in-vitro experimentation on suspension cultures of primary T-PLL cells, healthy CD3+ T cells, HH cells, and in co-culture experiments with NKtert cells. Cell suspensions were maintained at a density of 1.0×10⁶ cells/mL (primary T-PLL cells) and 2.5×10⁵ cells/mL (HH cells) for all cell culture experiments.

Cells were cultured in a HERAcell incubator (Thermo Scientific Heraeus) at 37° C. and 5% CO₂ with 90% humidity. CD4+ mature T-cell leukemia HH cells were originally isolated from a patient with Sezary Syndrome (Starkebaum et al., 1991). NKtert (human bone marrow stromal cells [BMSC]) were purchased from RIKEN Cell Bank in 2011. Only cells derived from the original cell stock as purchased, and which had been propagated for 2 to 3 passages before long-term storage in liquid nitrogen, were used. Cell cultures were terminated after the 10th passage (4 to 6 weeks of being in culture). Cells were authenticated following thawing by evaluation of characteristic growth behaviour and by flow cytometry. Cells were routinely tested for the presence of mycoplasma, using standard PCR protocols (primers: for1: 5′-acaccatgggagytggtaat-3′, (SEQ ID No; 1) rev1: 5′-cttcwtcgattycagacccaaggcat-3 (SEQ ID NO: 2)′, for2: 5′-gtgsggmtggatcacctcct-3 (SEQ ID NO: 3)′, rev2: 5′-gcatccaccawawacyctt-3′(SEQ ID NO: 4)).

Healthy CD3+ T-cells were isolated from healthy human donors.

For co-culture experiments human bone marrow stromal cells (BMSC) NKtert cells (RIKEN BRC, Japan) were seeded at concentrations of 1.5×10⁴ cells/well (96 well plate) and incubated at 37° C. in 5% CO₂. After 24 hours, NKtert cells at approximately 60-80% confluency were treated with 0.02 mg/mL Mitomycin C for 3 hours in RPMI-1460, and then washed twice with PBS (Life Technologies). After another 24 hours, 4×10⁵ T-PLL cells were added per well (with and without feeder cell support) and treated for 48 hours with the indicated compounds.

### In Vitro Drug Treatment and Cell Viability

EDO-S101 (EDO MundiPharma), and vorinostat (SAHA; SML0061-5 mg, Sigma-Aldrich) were dissolved in DMSO. The alkylating agent bendamustine (MundiPharma) was dissolved in methanol. Cells were treated with each compound (or compounds) at the indicated concentrations and times. Dosing was based on published ranges and IC₅₀/LD₅₀ titrations. Cell apoptosis was determined using dual staining for Annexin-V (AnxV) and 7AAD via flow cytometry.

Human primary T-PLL cells are unsuitable for cultivation under standard laboratory cell culture conditions, in part, due to their high levels of genomic heterogeneity and variable phenotypes. HH cells are derived from a highly chemotherapy resistant cutaneous lymphoma, and are suitable for cultivation under laboratory conditions. HH cells exhibit a comparable phenotype to T-PLL cells, and are therefore frequently used as a surrogate cell line for T-PLL cells for in vitro experiments. HH cells were therefore selected for the in vitro validation of EDO-S101.

### Murine Models

DBA2×C57B6JF1 mice were used as recipients in all experiments. Transplantable leukaemia/lymphoma cells derived from CD2-MTCP1p13 tg mice (Gritti et al, Blood 1998, 92, 268-73; blood, spleen, and bone marrow) were intraperitoneally injected into background-matched mice (to facilitate the generation of uniform cohorts). 1×10⁷ cells from CD2-MTCP1p13 mice were intraperitoneally injected into syngeneic recipients (n=26). Starting on day 10 post-transplantation, mice with a homogeneous distribution of leukemic blood leukocytes (WBC) were selected and randomly assigned into four treatment groups. Each group was then treated with either vehicle control (DMSO), fludarabine (34 mg/kg days 10, 15, 17, 21), bendamustine (day 10 at 60 mg/kg, days 15, 17, 21 at 20 mg/kg), and EDO-S101 (day 10 at 50 mg/kg, days 15, 17, 21 at 20 mg/kg) on the indicated days at the indicated doses.

Transplantable leukaemia/lymphoma cells derived from ΔJAK1 mice (Heinrich et al, Mol. Ther. 2013, 21, 1160-8; nodal/spleen mature T-cell lymphoma based on insertional mutagenesis activating JAK1) were intravenously injected into background-matched mice (to facilitate the generation of uniform cohorts). 2.5×10⁶ cells were transplanted intravenously into Rag1-deficient mice. Recipients of comparable leukocyte counts were then randomly divided into four treatment groups. Each treatment group was thenh treated with 18 mg/kg of either bendamustine, fludarabine, EDO-S101, or with vehicle control on days 7, 10, 13, 17, 22 (DMSO).

### Patient Samples

T-PLL cells were isolated from peripheral blood (PB) of T-PLL patients diagnosed according to WHO criteria (Swerdlow, S. H. et al Blood 2016, 127, 2375-90; Herling et al Blood 2004, 104, 328-335). Diagnosis was based on clinical features, immunophenotyping (flow-cytometry and histochemistry; including TCL1A/MTCP1 expression), FISH/karyotypes, and molecular studies (TCRmonoclonality). Human tumour samples were obtained under institutional review board (IRB)-approved protocols following written informed consent according to the Declaration of Helsinki. Collection and use was approved for research purposes by the ethics committee of the University Hospital of Cologne (#11-319). The patient cohort was selected based on uniform front-line treatment (87% of cases) with either single-agent alemtuzumab or fludarabine-mitoxantrone-cyclophosphamide (FMC) plus alemtuzumab chemoimmunotherapy as part of the TPLL120 (NCT00278213) and TPLL2 (NCT01186640, unpublished) prospective clinical trials or as included in the nation-wide T-PLL registry (IRB #12-146) of the German CLL Study Group. At diagnosis, patients had a median age of 62 years and included 1.5-times more men than women. FISH analysis used standard protocols (Vysis, Abbott).

### Western Blot Analysis

T-PLL cells were isolated from peripheral blood (PB) of T-PLL patients. T-PLL cells were cultured in suspension, and treated with either bendamustine (1 μM), vorinostat (1 μM), EDO-S101 (1 μM) or an equimolar combination of vorinostat/bendamustine (1 μM) for 36 hours at the indicated concentrations. After this time, the cells were harvested and lysed, the cell lysate sonicated, centrifuged to remove any cellular debris, and the supernatant collected. The protein concentration of each cell lysate solution (the supernatant) was determined and the western blot performed using standard methods.

The antibodies used were acHistone 3 (Sigma Aldrich), phospho-ATM Serine1981 (Sigma Aldrich), ATM (Sigma Aldrich), phospho-KAP-1 Serine824 (Sigma Aldrich), KAP-1 (Sigma Aldrich), phosphor-p53 Serine15 (Sigma Aldrich), acetyl-p53 (Sigma Aldrich), p53 (Sigma Aldrich), PARP (Sigma Aldrich), cleaved-PARP (Sigma Aldrich), and β-Actin (Sigma Aldrich).

### Example 1-Cell Viability

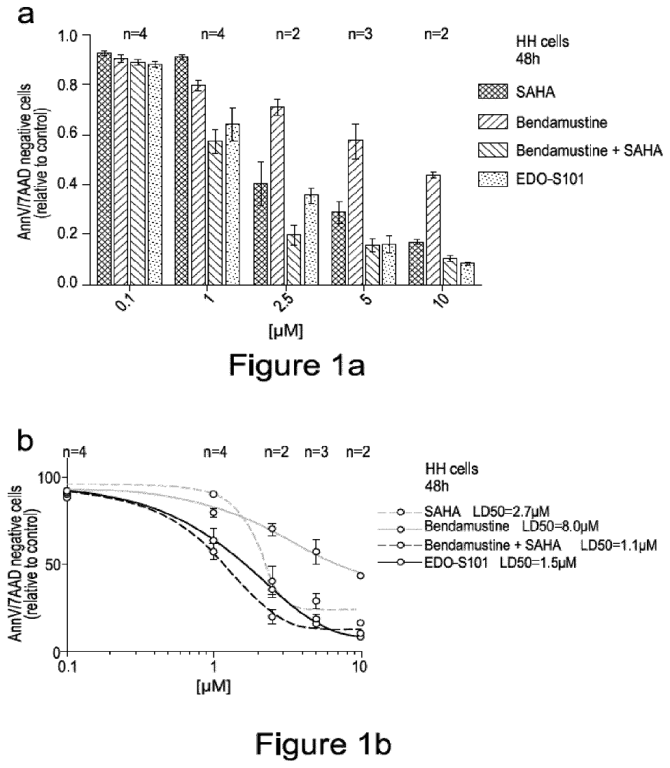

To evaluate the cytotoxicity of EDO-S101 in comparison to bendamustine and vorinostat (SAHA), HH cells were treated with either EDO-S101, bendamustine, vorinostat or an equimolar bendamustine/vorinostat combination over a period 48 hours. Cells were treated with either 0.1 μM, 1 μM, 2.5 μM, 5 μM or 10 μM solutions of the indicated compounds (FIG. 1a).

Following treatment, cell death was evaluated by staining cells with the apoptosis markers Annexin-V and 7-AAD, and the number of apoptotic cells quantified by flow cytometry. Annexin-V specifically targets and identifies apoptotic cells. 7-AAD is a marker of late stage apoptotic, or necrotic cells. The number of Annexin-V and 7-AAD negative cells was counted for each sample. Each experiment was repeated for the indicated number of times, and the average number of apoptosis negative cells plotted and normalised relative to an untreated control sample (FIG. 1a). A dose response curve (FIG. 1b) for each treatment was subsequently plotted, and the LD₅₀ (median lethal dose) of each treatment determined.

The equimolar combination of bendamustine and vorinotstat (LD₅₀: 1.1 μM), and EDO-S101 (LD₅₀: 1.5 M) demonstrated marked potency in HH cell death induction after 48 hours of treatment. Both the bendamustine/vorinostat combination and EDO-S101 exhibited LD₅₀ values in the low micromolar range. Furthermore, both EDO-S101 and the bedamustine/vorinostat combination treatment demonstrated enhanced cytotoxicity compared to vorinostat (LD₅₀: 2.7 μM) and bendamustine (LD₅₀: 8.0 μM) as single agents.

### Example 2-Western Blot Analysis of Patient T-PLL Samples

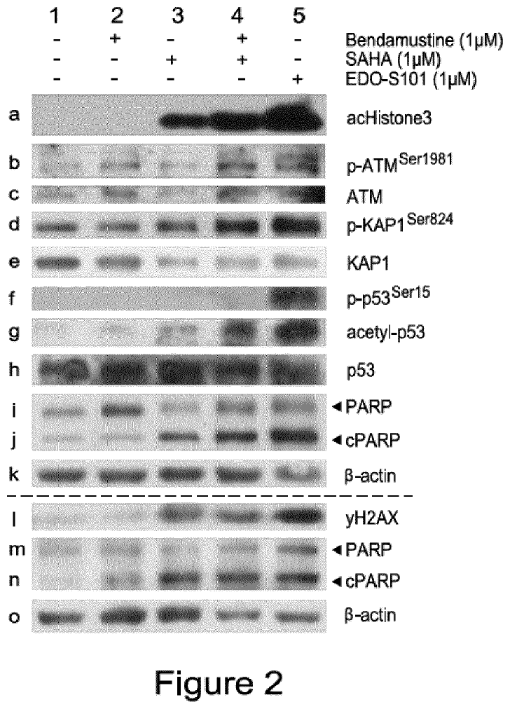

T-PLL cells (ATM mutated at L1238, mono-allelic ATM loss, copy no=1.41) were isolated from peripheral blood (PB) of T-PLL patients were cultured in suspension, and treated with 1 μM of either bendamustine (FIG. 2, lane 2), vorinostat (lane 3), EDO-S101 (lane 5) or an equimolar combination of vorinostat/bendamustine (lane 4) for 36 hours. After this time, the cells were harvested, lysed and protein expression levels determined by western blot analysis.

FIG. 2 shows western blots of the cell lysate for each treatment, compared to a negative control (lane 1). Staining for β-Actin was used as a loading control for each western blot ran (rows a to k, and rows l to o respectively).

HDAC inhibitors target proteins which promote the deacetylation of histones, or the deacetylation of other proteins. Acetylation and deacetylation of histones are post-translational modifications implicated in DNA replication and repair, and therefore the acetylation status of histones is crucial in cell replication pathways. Inhibition of HDAC activity is therefore linked to induction of the DNA damage response. HDAC inhibitors used in these experiments include vorinostat and the fusion molecule EDO-S101.

DNA alkylating agents prevent normal DNA replication pathways from functioning by binding to DNA, and therefore cause replication stress. In an attempt to repair the damage caused, the cell recruits an array of proteins, in what is referred to as the DNA Damage response (DDR). Many of the proteins recruited in the DDR may be used as biomarkers for damage and replication stress. Such biomarkers include increased expression levels of γH2AX, phosphorylated ATM (pATM), phosphorylated Kap1 (pKap1), and stabilisation of p53. DNA alkylating agents used in this example include bendamustine, and the fusion molecule EDO-S101 (which is also a HDAC inhibitor).

Referring to FIG. 2, it is clear that treatment of T-PLL cell samples isolated from patients with EDO-S101 led to the most significant induction of the DDR, compared to treatment with bendamustine, vorinostat or a combination thereof. Cells treated with EDO-S101 revealed the largest increase in levels of γH2AX (row l), pATM (row b), and pKAP1 (row d), all of which are heavily implicated in the DNA damage response. In line with the induction of the DDR, the expression levels of Kap1 (row e) were reciprocally related to the levels of pKap1. These results indicate that EDO-101 exhibits the most potent DNA alkylating activity compared to vorinostat and bendamustine, and furthermore, that EDO-S101 exceeds the potency of a combination of vorinostat and bendamustine.

The induction of the DDR also causes stabilization of p53 (row h) and subsequent phosphorylation (row f) and acetylation of p53 (acetyl-p53; row g). Referring to FIG. 2, treatment of cells with EDO-S101 resulted in the greatest accumulation of acetyl-p53 (row g) and p-p53 (row f), compared to bendamustine, vorinostat or a combination thereof. These results further support EDO-S101 being the most potent inducer of DNA damage.

Where DNA damage is extensive and the DNA cannot be repaired, p53 pathways are responsible for inducing cell apoptosis. One such pathway is characterized by the cleavage of PARP. As can be seen in FIG. 2, treatment of cells with EDO-S101 caused the greatest increase in cleaved PARP (cPARP; row j), compared to bendamustine, vorinostat, or a combination thereof. These data indicate that treatment with EDO-S101 caused the most extensive and irreparable DNA damage in T-PLL cells, promoting cell apoptosis. Furthermore, these data are indicative that treatment of T-PLL cells from patients with EDO-S101 effectively overcomes the protective effect conferred by stromal cells against cell apoptosis.

Referring to FIG. 2, treatment of T-PLL cells with EDO-S101 resulted in the largest increase in acetylation of histone3 (acHistone3), compared to vorinostat or a combination of vorinostat and bendamustine. These data indicate that EDO-S101 was a more effective HDAC inhibitor than vorinostat alone, or vorinostat in combination with bendamustine, in T-PLL cells.

In conclusion, FIG. 2 indicates that EDO-S101 induces the strongest DNA damage response in cells. This can be attributed to its enhanced potency as a DNA alkylator, and also as a HDAC inhibitor, compared to bendamustine or vorinostat. Furthermore, it is clear that the DNA damage and hyperacetylation induced by EDO-S101, exceeds that induced by a combination of vorinostat and bendamustine (see for example the comparably elevated levels of pATM, acetyl-p53, pKAP1, γH2AX and acHistone 3). As a result, apoptosis in strongly induced in EDO-S101 treated cells, compared to those treated with a combination of vorinostat and bendamustine (see elevated levels of cPARP).

### Example 3-Induction of Apoptosis and Resistance of EDO-S101 Treated Cells to Stromal Cell Mediated Protection in T-PLL

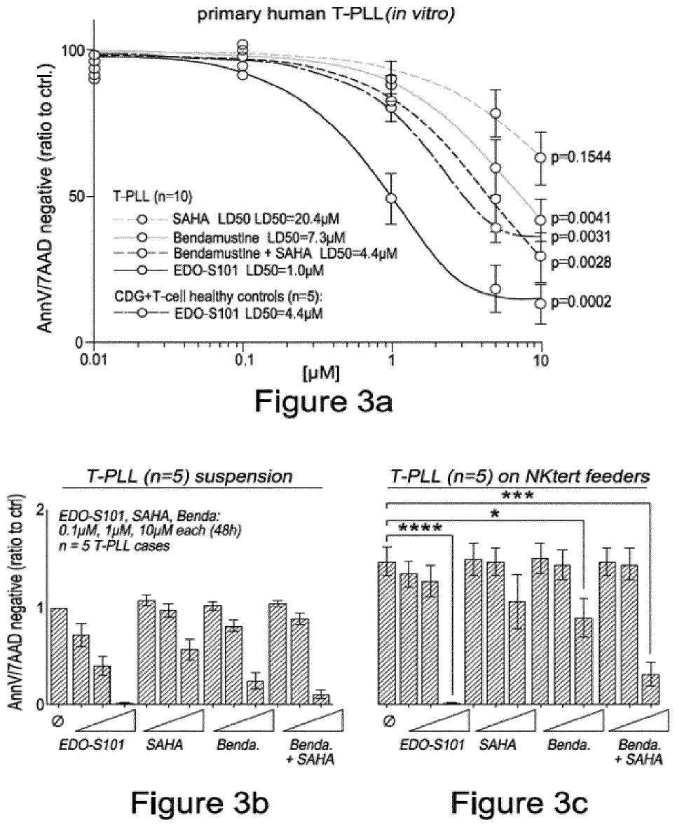

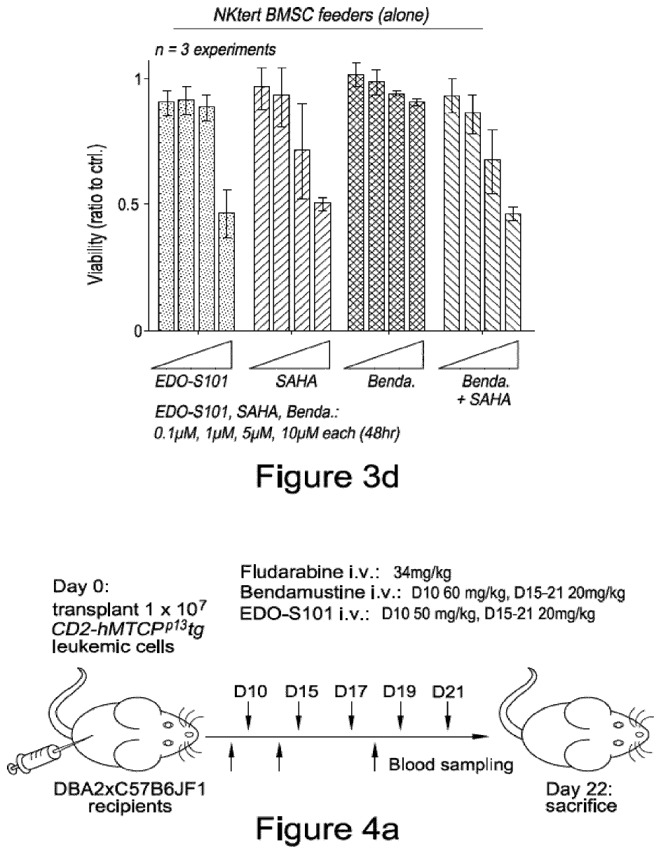

To further evaluate apoptosis in cells treated with EDO-S101, primary human T-PLL cells were treated with either vorinostat, bendamustine, EDO-S101, or an equimolar combination of vorinostat and bendamustine, at 0.01 μM, 0.1 μM, 1 μM, 5 μM or 10 μM concentrations, and incubated for 48 hours. Following treatment, cell death was evaluated by staining cells with the apoptosis markers Annexin-V and 7-AAD, and the number of apoptotic cells quantified by flow cytometry. Each experiment was repeated for the indicated number of times, and the average number of apoptosis negative cells plotted and normalised relative to an untreated control sample. A dose response curve (FIG. 3a) for each treatment was subsequently plotted, and the LD₅₀ (median lethal dose) of each treatment determined.

The LC₅₀ values for each treatment were calculated for vorinostat (20.4 μM), bendamustine (7.3 μM), EDO-S101 (1.0 μM), or an equimolar combination of vorinostat and bendamustine (4.4 μM). The LC₅₀ value for EDO-S101 was found to be lower in primary human T-PLL cells (1.0 μM) (FIG. 3a) than in HH cells (1.5 μM) (FIG. 1b) under comparable experimental conditions indicating enhanced efficacy against T-PLL cells. Furthermore, EDO-S101 (1.0 μM) exhibited approximately a 4-fold increase in potency against T-PLL cells compared to a combination of vorinostat and bendamustine (4.4 μM).

The LC₅₀ of EDO-S101 in healthy CD3+ T-cells was determined to be 4.4 μM, indicating that EDO-S101 was approximately 4-fold more potent against T-PLL cells compared to healthy CD3+ T-cells, under experimental conditions. This result demonstrated that EDO-S101 had selectivity for T-PLL cells over healthy T-cells.

NKtert bone-marrow stromal cells are known to protect mutated T-cells against the effects of drugs and against apoptosis. To evaluate the protections conferred by NKtert cells to primary human T-PLL cells, NKtert cells and T-PLL cells were co-cultured for treatment with EDO-S101. Primary T-PLL cells with (FIG. 3b) and without (FIG. 3c) co-cultures of NKtert cells were treated with increasing concentrations (0.1, 1, or 10 μM) of vorinostat, bendamustine, an equimolar combination of vorinostat and bendamustine, or EDO-S101, and incubated for 48 hours.

Following treatment, cell death was evaluated by staining cells with the apoptosis markers Annexin-V and 7-AAD, and the number of apoptotic cells quantified by flow cytometry. Each experiment was repeated for the indicated number of times, and the average number of apoptosis negative cells plotted as a ratio relative to an untreated control sample (FIG. 3b, FIG. 3c).

Referring to FIG. 3b, the control sample Ø on the left hand graph is normalised to 1 for normal T-PLL cells. As can be seen from FIG. 3c the control sample Ø for the T-PLL/NKtert co-cultured cells is greater than 1, indicating enhanced survival of T-PLL cells in the presence of NKtert cells compared to monoculture.

Referring to FIGS. 3b and 3c, both T-PLL cells, and T-PLL/NKtert co-cultured cells, were sensitive to treatment with EDO-S101. Extensive apoptosis was observed in both T-PLL cells, and co-cultured T-PLL/NKtert cells, when treated with 10 μM EDO-S101, with an approximate cell death count of greater than 95%. These data indicate that treatment of T-PLL cells with EDO-S101 overcame the protection conferred by the NKtert cells. Furthermore, T-PLL cells treated with either bendamustine, vorinostat, or an equimolar combination of bendamustine and vorinostat, were not as effective as EDO-S101 in overcoming NKtert associated protection co-cultured T-PLL cells. These data are further supported by the observation that treatment of human T-PLL cells with EDO-S101 led to the most enhanced levels of cPARP, a key indicator of cell apoptosis (FIG. 2).

It was hypothesised that EDO-S101 could be affecting the viability of the NKtert cells in the co-cultured T-PLL/NKtert cell experiments shown in FIG. 3c. Consequently, the effect on cell viability of NKtert bone marrow stromal cells (BMSC) feeders cells alone was also investigated. Cells were treated with either bendamustine, vorinostat, an equimolar combination of bendamustine and vorinostat, or EDO-S101, at either 0.1, 1, 5 or 10 μM concentrations, incubated for 48 hours, and cell viability assessed using MTT assays (FIG. 3d). As can be seen in FIG. 3d, the reduction in viability of NKtert cells treated with EDO-S101, vorinostat, or an equimolar combination of vorinostat and bendamustine, were largely comparable over the concentrations investigated. Bendamustine treatment did not have a pronounced concentration dependent effect on the viability of NKtert cells. Importantly, the viability of cells treated with EDO-S101 and vorinostat at 10 μM were comparable, providing confidence that the cell death induced by treatment of T-PLL/NKtert co-cultured cells with EDO-S101 (FIG. 3c) was not a result of reduced viability of NKtert cells (FIG. 3d).

### Example 4-In Vivo Analysis of Leukemic Blood Leukocytes (WBC) Count Following Treatment with EDO-S101

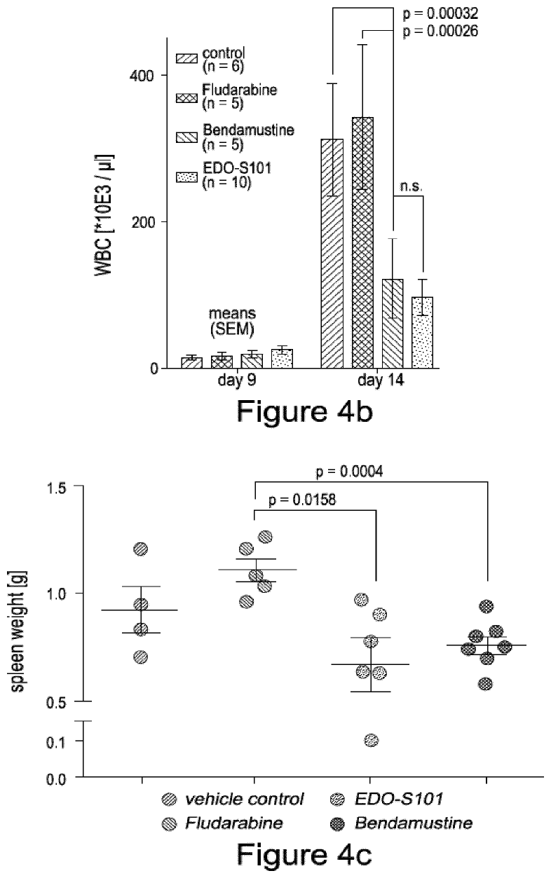

Mice were injected with leukaemia cells derived from CD2-MTCP1 mice, as previously described (FIG. 4a).

CD2-MTCP1 cells are an aggressive, transplantable subline and are suitable for in vivo analysis as a T-PLL like model. At day 10 post-transplantation of CD2-MTCP1 cells by intraperitoneal injection, mice with comparable leukemic blood leukocytes (WBC) counts were divided into four groups at random. Each group was intravenously administered either fludarabine (34 mg/kg), bendamustine (Day 10, 60 mg/kg; Day 15-21, 20 mg/kg) or EDO-S101 (Day 10, 50 mg/kg; Day 15-21, 20 mg/kg) at the indicated doses on Day 10, Day 15, Day 17, Day 19 and Day 21 post-transplantation. Samples of blood were taken at regular intervals (Day 9 and Day 14 post-transplantation), and the mice sacrificed at 22 days post-transplantation (FIG. 4a).

Fludarabine was selected for experiments as a comparative compound, and is a FDA approved chemotherapy for the treatment of leukemia and lymphoma. Fludarabine is a purine derivative, and interferes with the replication of DNA. It is on the World Health Organisation's List of Essential Medicines.

The blood samples taken were analysed for leukemic blood leukocyte levels (WBC), and the average cell count in each group determined. Comparison of WBC count at Day 14 and Day 9, revealed that bendamustine and EDO-S101 significantly delayed the increase in WBC cells compared to a control sample and fludarabine (FIG. 4b). These data indicated that EDO-S101 and bendamustine was delaying the onset of disease progression in the recipient mice.

Following sacrifice of the mice on day 22 post-transplantation, the post-mortem spleen weights of each mouse were determined (FIG. 4c). The reduced average spleen weight in mouse cohorts treated with bendamustine or EDO-S101, compared to cohorts treated with fludarabine or a control, corroborate the findings previously discussed in FIG. 4b. Tumour manifestation was therefore shown to be less advanced in cohorts treated with EDO-S101 or single-agent bendamustine, compared to fludarabine treated or control cohorts.

### Example 5-In Vivo Analysis of Leukemic Blood Leukocytes (WBC) Count Following Treatment with EDO-S101

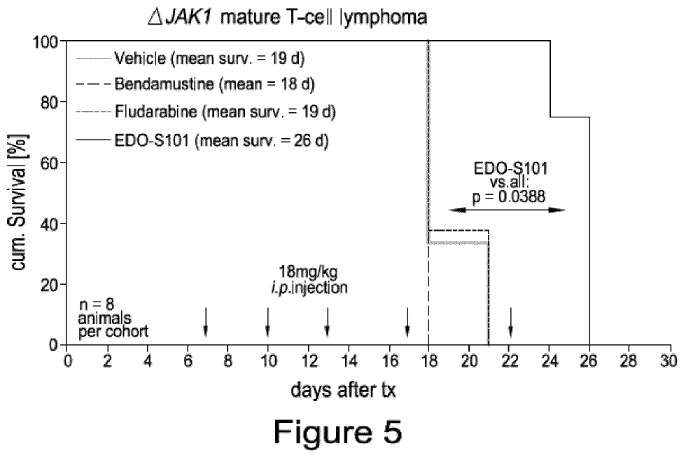

Mice were injected with leukaemia/lymphoma cells derived from ΔJAK1 mice, as previously described (FIG. 5). ΔJAK1 is a model for mature T-cell lymphoma. Mice were divided into four groups at random post-transplantation. Each group was intravenously administered either fludarabine (18 mg/kg), bendamustine (18 mg/kg) or EDO-S101 (18 mg/kg) at the indicated doses on Day 7, Day 10, Day 13, Day 17 and Day 22. The percentage survival of each cohort was plotted as a function of time (FIG. 5). The overall survival (mean survival 26 days) of mice treated with EDO-S101 was found to be significantly prolonged compared to control mice (mean survival 19 days), bendamustine (mean survival 18 days) or fludarabine (mean survival 19 days). These data indicate that treatment with EDO-S101 has a positive effect on the overall survival of mice with T-cell lymphoma, increasing the average survival time by as much as 8 days compared to bendamustine or fludarabine.

1.-11. (canceled)

12. A method of treating T-cell prolymphocytic leukemia (T-PLL) in a patient in need thereof, comprising administering to said patient a composition comprising an effective amount of tinostamustine or a pharmaceutically acceptable salt thereof.

13. The method of claim 12, wherein the composition is in a liquid or solid form.

14. The method of claim 13, wherein the composition comprises a liquid carrier selected from polyethylene glycol, cyclodextrin and a fatty oil, and a combination thereof.

15. The method of claim 14, wherein the composition is a solution or a suspension.

16. The method of claim 15, wherein the method comprises parenterally administering the composition to said patient.

17. The method of claim 16, wherein the parenteral administration is an intradermal, intramuscular, intraperitoneal, intravenous, subcutaneous, intranasal, epidural, intranasal, intracerebral, intraventricular, intrathecal, intravaginal or transdermal administration.

18. The method of claim 16, wherein the parenteral administration is an intravenous administration.

19. A kit comprising a composition comprising a therapeutically effective amount of tinostamustine or a pharmaceutically acceptable salt thereof, together with instructions for treating T-cell prolymphocytic leukemia (T-PLL) in a patient in need thereof, wherein said therapeutically effective amount is an amount for administration to the patient at a dosage level effective for treating T-PLL.

20. The method of claim 19, wherein the composition is in a liquid or solid form.

21. The kit of claim 20, wherein the composition comprises a liquid carrier selected from polyethylene glycol, cyclodextrin and a fatty oil, and a combination thereof.

22. The kit of claim 21, wherein the composition is a solution or a suspension.

23. The kit of claim 19, wherein the composition is suitable for parenteral administration.

24. The method of claim 23, wherein the parenteral administration is an intradermal, intramuscular, intraperitoneal, intravenous, subcutaneous, intranasal, epidural, intranasal, intracerebral, intraventricular, intrathecal, intravaginal or transdermal administration.

25. The method of claim 23, wherein the parenteral administration is an intravenous administration.
